(19) United States
(12) Patent Application Publication
(10) Pub. No.: US 2016/0158501 A1
(43) Pub. Date: Jun. 9, 2016

(54) PERCUTANEOUS SCALPEL AND TISSUE DILATOR

(71) Applicant: David Farris, Portland, OR (US)

(72) Inventor: David Farris, Portland, OR (US)

(21) Appl. No.: 14/959,913

(22) Filed: Dec. 4, 2015

Related U.S. Application Data
(60) Provisional application No. 62/087,556, filed on Dec. 4, 2014.

(51) Int. Cl. A61M 25/06; A61M 29/02; A61M 25/09; A61B 17/3211

(52) U.S. Cl. CPC A61M 25/0606; A61B 17/3211; A61M 29/02; A61M 25/09041; A61M 25/0693

(57) ABSTRACT

Embodiments herein provide a combined percutaneous scalpel and tissue dilator. Embodiments herein also improve (a) the process, and (b) the result of placement of percutaneous vascular access catheters and other similar devices by providing a combined scalpel-dilator that cuts with minimal tissue disruption the percutaneous access tract necessary for a catheter or other device.

### TECHNICAL FIELD

Embodiments herein relate to the field of medical devices, and, more specifically, to a percutaneous scalpel and tissue dilator.

### BACKGROUND

Current percutaneous device insertion/placement causes the formation of a large opening in the skin. The separate scalpels and dilators used in such methods create unnecessary tissue trauma that could be prevented or limited with a new device and method.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 is an example of an incision in skin created by a scalpel following a known method of placing a percutaneous device.

FIG. 2 is an example of an incision in skin created by a scalpel following a known method of placing a percutaneous device.

FIG. 3A shows a side view of an example scalpel-dilator in accordance with embodiments herein.

FIG. 3B shows a top down view of an example scalpel-dilator in accordance with embodiments herein.

FIG. 4A shows a top down view of an example scalpel-dilator in accordance with embodiments herein.

FIG. 4B shows a side view of an example scalpel-dilator in accordance with embodiments herein.

FIG. 5 shows a side view of an example scalpel-dilator in accordance with embodiments herein.

FIG. 6A shows a side view of an example scalpel-dilator in accordance with embodiments herein.

FIG. 6B shows a top down view of an example scalpel-dilator in accordance with embodiments herein.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Embodiments herein provide a combined percutaneous scalpel and tissue dilator, which at times is referred to herein as a combination scalpel-dilator or a scalpel-dilator. Embodiments herein also improve (a) the process, and (b) the result of placement of percutaneous vascular access catheters and other similar devices by providing a combined scalpel-dilator that cuts with minimal tissue disruption the percutaneous access tract necessary for a catheter or other device in a manner that (1) combines two steps in the current, standard procedure into one step, (2) is protective of the involved structures, (3) is fitted to the device (minimizing bleeding and infection risk), and (4) leaves only a small scar.

In an example technique for percutaneous placement of a cannula into a blood vessel, such as the Seldinger technique, the following are steps typically undertaken.

(1) In a sterile environment, the target vessel is entered percutaneously with a needle and syringe; aspirating blood into the syringe confirms the needle tip is in the vessel lumen.

(2) The syringe is removed and a soft-tipped guide wire is placed through the needle lumen into the lumen of the vessel and advanced well past the tip of the needle. The wire is of sufficient length that the back end of the wire remains in hand.

(3) The wire is held in situ as the needle is withdrawn back over the full length of the wire and discarded.

(4) A scalpel is laid against the wire where it enters the skin and advanced, incising the skin and subcutaneous fascia to create an opening for the dilator.

(5) A tapered dilator—generally hard plastic—is advanced forcibly over the wire to open the tract necessary for the device.

(6) The wire is held in place as the dilator is removed and discarded.

(7) The cannula is advanced over the wire until it is at the desired depth; the back end of the wire is now protruding from the back end of the cannula.

(8) The cannula is held in place as the wire is removed and discarded.

(9) Air is aspirated from the cannula, and then flushed with a neutral solution, e.g., sterile saline.

(10) The cannula is capped and secured (e.g., sutured.)

While the Seldinger technique is referenced above, the present embodiments are applicable to other, non-blood vessel applications.

In an embodiment herein, the method outlined above is modified by combining steps 4 and 5. Namely, the skin and subcutaneous fascia are incised with a scalpel-dilator, as described herein. The scalpel-dilator has a hollow center and goes over the wire just as does the conventional dilator. Within the taper of the tip, however, are sharp edges that incise the skin and subcutaneous fascia, thereby eliminating the separate scalpel step noted above. Further, the incision/opening is made only wide enough to allow passage of its own second stage, the dilator.

Disclosed herein is a method of percutaneous placement of a cannula or other device into a blood vessel. In embodiments, the disclosed method includes percutaneously entering a target vessel, such as a blood vessel, of as subject, such as a human or veterinary subject, with a needle and connected syringe. The syringe is removed and a guide wire, typically a soft tip guide wire, is passed through the needle lumen into the lumen of the vessel and advanced past the tip of the needle. The needle is withdrawn over the wire while the wire is held in place relative to the vessel and discarded. A disclosed combination scalpel-dilator is then passed over the wire and forcibly applied to the tissue to open the tract for the cannula or other device, incising the skin and subcutaneous fascia to create an opening for the cannula or other device. Once satisfied with the tract, the combination scalpel-dilator is removed and discarded while the wire is held in place relative to the vessel. To complete placement of the cannula or other device, the cannula or other device is passed over the wire until it is at the desired depth within the tissue and the wire removed and discarded. Certain embodiments of the method include aspirating air from the cannula or other device and then flushing with a neutral solution, for example to make sure that the line is clear and clean. Certain embodiments of the method include securing the cannula or other device to the subject to prevent movement of the cannula or other device relative to the blood vessel and/or subject. Certain embodiments of the method include capping the cannula or other device, for example to prevent unwanted substances from entering the cannula or other device, or attaching the cannula or other device to secondary tubing. Certain embodiments of the method include aspirating blood into the syringe to confirm the needle tip is in the vessel lumen, for example by applying negative pressure to the syringe while penetrating the vessel.

As indicated by the above description, the current method includes excess steps that are streamlined using the present methodology. Further, the incision (step 4, above) is generally done with a triangular (#11) blade. The width of the incision in the skin is often 2-3 times the diameter of the final device being inserted. This exposes subcutaneous tissue, thereby increasing infection risk, and creates a scar larger than necessary. Past the skin, advancement of the triangular blade is blind. Absent luck, the incision is either deeper or shallower than ideal. In the former instance, the wall of the vein is incised significantly more than the diameter of the final device. This leads to bleeding. In the latter instance, advancement of the hard plastic dilator requires significant force. This causes local tissue trauma which may be associated with infection and scarring. In addition, by combining the scalpel and dilator the disclosed method reduces the amount of medical waste that needs to the processed after it is discarded.

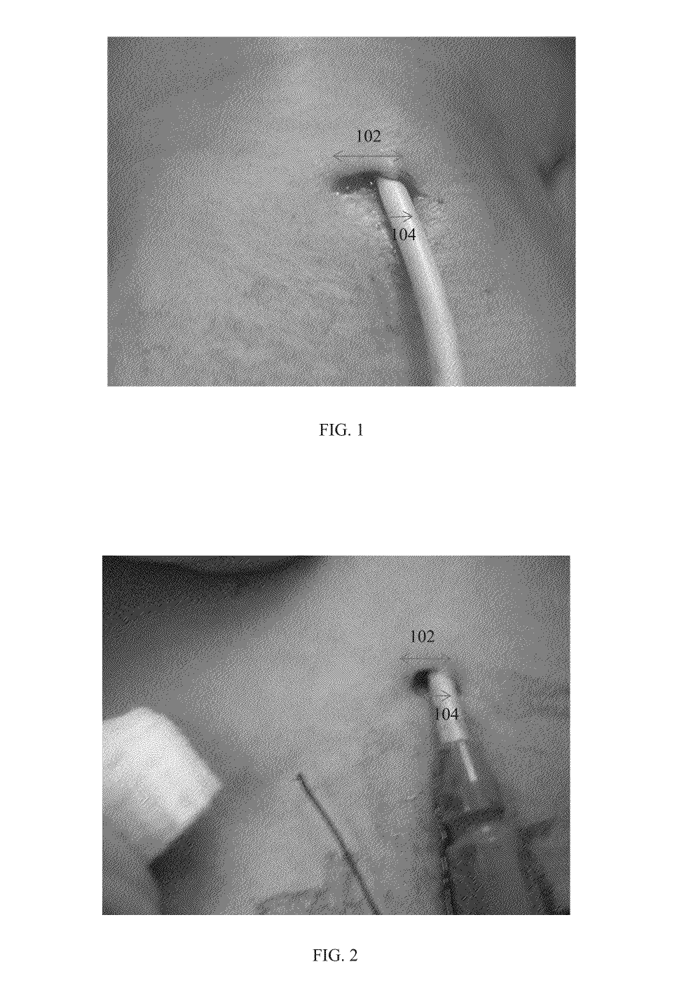

FIGS. 1 and 2 are examples of incisions in skin created by a scalpel following a known method. As seen in the figures, the incision has an excess width 102 relative to the width 104 of the final device (in this case a catheter).

Disclosed herein is a scalpel-dilator. In certain embodiments, a scalpel-dilator includes an elongated hollow tubular portion; a tip portion having an end, where the tip portion tapers toward the end forming a tapered end; two or more grooves in the tapered end; and one or more ridges between the two or more grooves. In certain embodiments, at least two or more of the grooves are placed adjacent to one another, so that the ridge formed between grooves terminates at a point at the tapered end of the scalpel-dilator. Such a point has the advantage of being able to pierce and/or cut the tissue, such as the skin and/or blood vessels of a subject, for example during placement of the scalpel-dilator. As the device is advanced over a guide wire and enters the skin, the tapered tip will stretch the tissue and the sharp edge will cut it, allowing the device easy entrance. Thus, the device provides an advantage of both cutting and dilating. In an embodiment, a scalpel-dilator is a single piece, hard plastic, metal, or a combination thereof with grooves cut into one side of its tapered tip such that a sharp, pointed ridge is created between the grooves. In certain embodiments, a scalpel-dilator has between 2 and 10 grooves, although an additional number beyond 10 could be used in the certain applications. In certain embodiments, the scalpel-dilator has two grooves and one ridge. In certain embodiments, the grooves of the scalpel-dilator include a leading deep portion at the tip of the tapered end and a more shallow trailing portion running away from the tapered end. In certain embodiments each of the two or more grooves are the same length. In other embodiments at least one of the two or more grooves is a different length than the other groove or grooves, for example only one groove can be a different length than the others or all the or some of the grooves can be of different lengths than others. In certain embodiments each of the two or more grooves are the same depth. In other embodiments at least one of the two or more grooves is a same depth than the other groove or grooves, for example only one groove can be a different depth than the others or all the or some of the grooves can be of different depths than others.

The cutting efficiency of the sharp ridge may be improved by adding serrations to the scalpel-dilator. Thus, in certain embodiments, a scalpel-dilator further includes one or more serrations, such is between 1 and 10 in at least one of the one or more ridges.

The taper terminates with an end. This end can be perpendicular to the axis of the scalpel-dilator or it can have an angle, such as an angle of between about 0° and about 30° from the perpendicular, although greater angles could be used.

In embodiments, the back end of the dilator will vary according to the application. In many dilators currently used, for instance, the back end is a standard Luer-type connector allowing connection with a standard syringe. Thus, in certain embodiments, the scalpel-dilator includes a Luer-type connector on the tubular portion of the scalpel-dilator opposite the tapered end.

By replacing the existing scalpel with a cutting device matched to the final catheter (or other device), a variety of benefits are realized. Two major steps of the prior process are combined, improving efficiency of the procedure. The tissue tract matches the final device, minimizing bleeding and exposure of subcutaneous tissue. In current practice, tissue beyond the reach of the scalpel is divided by blunt force, creating damage both longitudinally and radially. The disclosed device minimizes this trauma, improving tissue healing and decreasing infection risk. And, scarring is minimized. Therefore, in certain embodiments, the outside diameter (OD) of a final dilating section of the combination scalpel-dilator is marginally smaller than the OD of a desired final device to be inserted into the tissue of a subject.

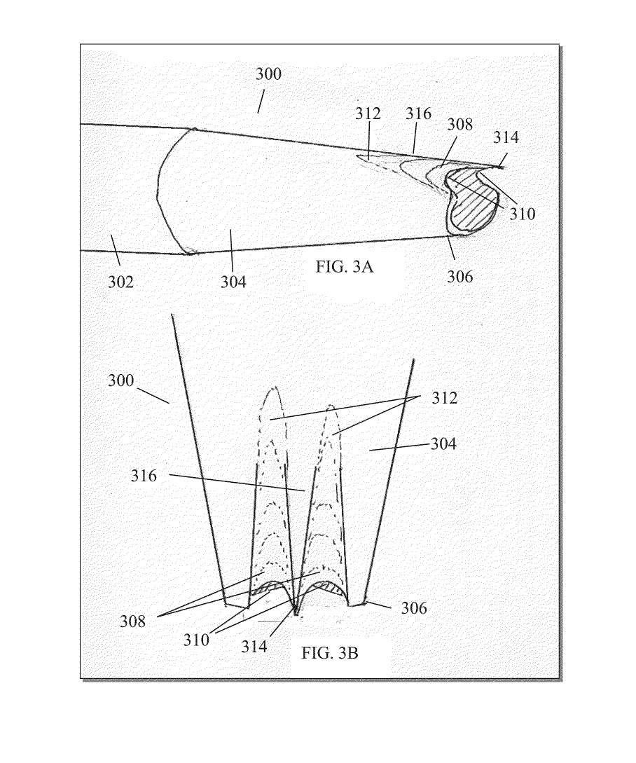

FIGS. 3A and 3B show an example scalpel-dilator 300 in accordance with embodiments herein. Scalpel-dilator 300 has an elongate, tubular portion 302 and a tip 304. While a substantially circular cross section of tube is shown it is envisioned that non-circular tubing, such as ovoid tubing could be used. As shown, tip 304 is tapered toward end 306 such a taper can be a straight taper or gradual, for example an arc of constant or variable circumference, additionally the joint between the tubular portion 302 and tip 302 can be rounded over as opposed to the sharp joint as shown. At end 306, there are two grooves 308 formed in tip 304. Grooves 308 each have a leading deep portion 310 and a more shallow trailing portion 312 as the groove tapers. Grooves 308 can be parallel (or substantially parallel), divergent or convergent. Between the grooves 308, there is formed a point 314 that aids in the piercing/cutting action of the scalpel-dilator 300 during insertion. Point 314 is the leading portion of a ridge 316 that extends between the grooves 308, which is the unmodified tip 304 residing between the grooves.

In an alternative embodiment, cutting efficiency of the sharp ridge may be improved by adding serrations to the scalpel-dilator.

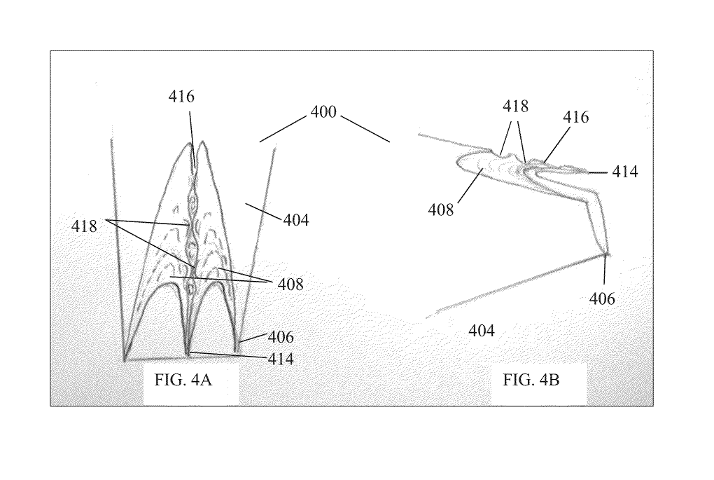

FIGS. 4A and 4B show an example scalpel-dilator 400 in accordance with embodiments herein, wherein the sharp ridge includes serrations. Scalpel-dilator 400 has a tip 404. As shown, tip 404 is tapered toward end 406. At end 406, there are two grooves 408 formed in tip 404. Between the grooves 408, there is formed a point 414 that aids in the piercing/cutting action of the scalpel-dilator 400 during insertion. Point 414 is the leading portion of ridge 416. Along ridge 416 are provided one or more serrations 418 to increase cutting efficiency.

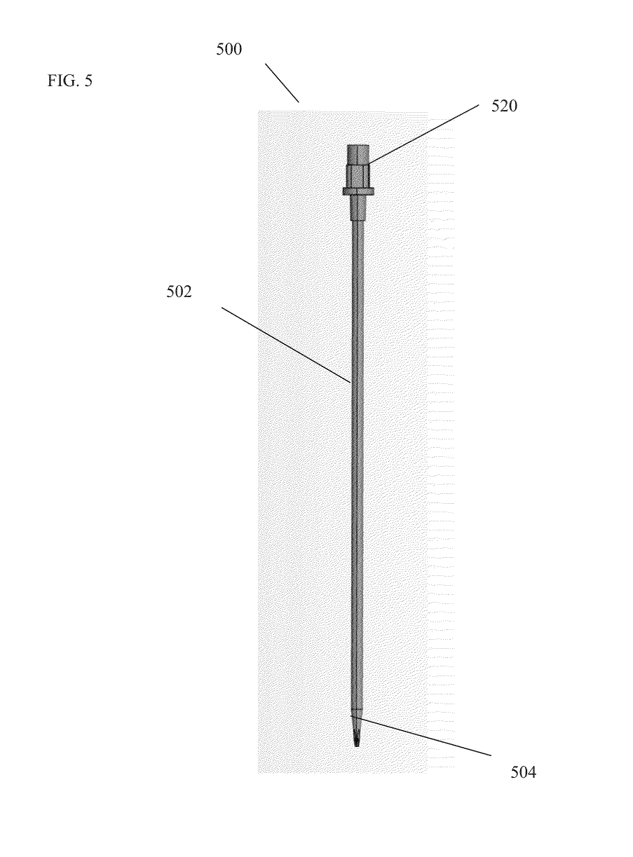

FIG. 5 shows an example scalpel-dilator 500 in embodiments herein. Scalpel-dilator 500 has an elongate, tubular portion 502 and a tip 504. In the embodiment shown, tube portion 502 terminates opposite tip 504 with connector 520, such as a Luer-type connector.

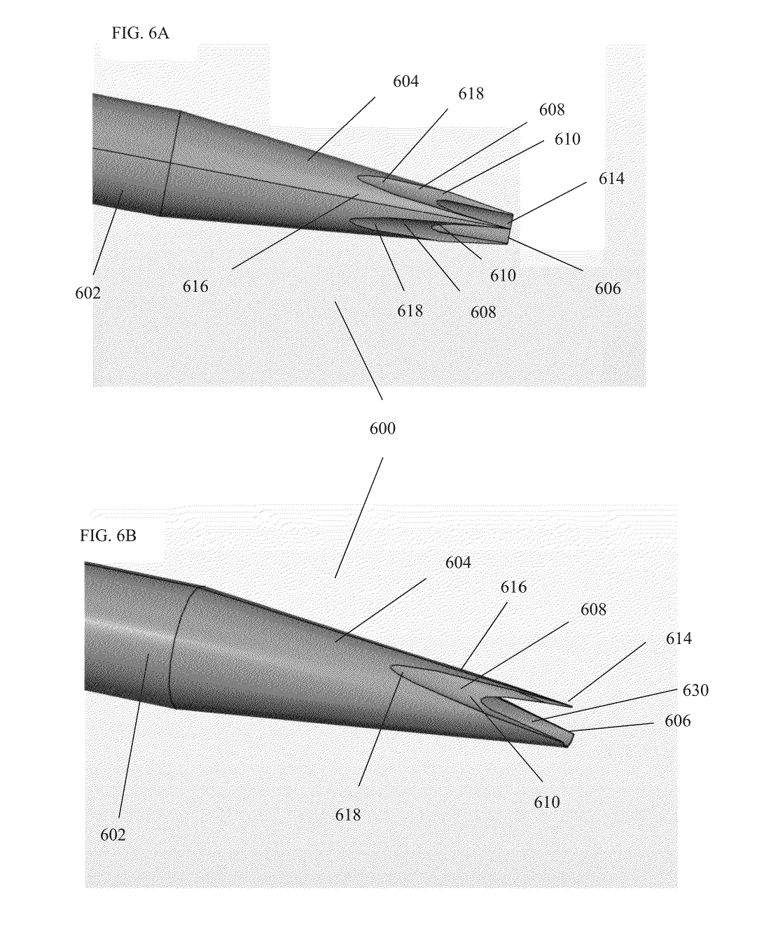

FIGS. 6A and 6B show an example scalpel-dilator 600 in accordance with embodiments herein. Scalpel-dilator 600 has an elongate, tubular portion 602 and a tip 604. As shown, tip 604 is tapered toward end 606 such a taper can be a straight taper or it can be a gradual curve, for example an arc of constant or variable circumference, additionally the joint between the tubular portion 602 and tip 602 can be rounded over as opposed to the sharp joint as shown. At end 606, there are two grooves 608 formed in tip 604. Grooves 608 each have a leading deep portion 610 and a more shallow trailing portion 612 as the groove tapers. Grooves 608 can be parallel (or substantially parallel), divergent or convergent. Between the grooves 608, there is formed a point 614 that aids in the piercing/cutting action of the scalpel-dilator 600 during insertion. Point 614 is the leading portion of a ridge 616 that extends between the grooves 608, which is the unmodified tip 604 residing between the grooves. Is shown, end 606 is shown as perpendicular to the central axis of tip 604 and/or tubular portion 602. The angle can be changed, for example as shown in FIGS. 3A-3B and 4A-4B. Scalpel-dilator 600 includes inner diameter 630, which can be sized to just slip over the guide wire (not shown).

While two grooves are shown in the figures noted above, in other embodiments, 3, 4, 5 or more grooves may be provided in a scalpel-dilator whether each are the same or different in length and/or depth.

In other embodiments, a small metal blade or blades may be implanted in the metal or plastic tube to enhance cutting, for example the tapered section of the disclosed scalpel-dilator can be paired with a tubular section, such as by embedding a metal tapered section into a plastic tubular section.

In an embodiment, the cutting point, grooves, and/or ridge may not need to be scalpel-sharp to accomplish the cut, but rather “plastic-sharp,” given that a stretched tissue will divide more easily than one relaxed. The term “plastic-sharp” refers to a edge that can be molded or formed into a plastic structure forming a sharp cutting surface. While it is envisioned that hard plastic will be functionally adequate and most cost effective to manufacture, the proposed configuration could be made of another suitable material, e.g., stainless steel. In certain embodiments the grooves and/or taper are formed in a preexisting tube section, for example by cutting, machining, stamping, or other method that produces the final product. In certain embodiments, the grooves and/or taper is formed by casting, molding, stamping, extrusion, or some other method, such that cutting and the like is not required to form the grooves and/or taper.

In embodiments, a scalpel-dilator is adaptable to virtually any size desired and useful in any procedure involving dilation over a wire, e.g. dilation of urethral strictures, tube thoracostomies (chest tubes), emergency airway devices (e.g., needle crico-thyroid-otomies), and any procedure involving advancing a cannula or other tube-like device over a guide wire.

Further disclosed is a kit for percutaneous placement of a cannula or other device. Such a kit includes a scalpel-dilator, typically in sterile packaging, for example as a single use item. In certain embodiments, a kit further includes one or more of a guide wire, a needle, a syringe and/or a cannula, or other device to be implanted in a subject, wherein the cannula or other device are sized with the combination scalpel-dilator.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

1. A combination scalpel-dilator, comprising:
an elongated hollow tubular portion;
a tip portion having an end, the tip portion tapering toward the end forming a tapered end;
two or more grooves in the tapered end; and
one or more ridges between the two or more grooves.

2. The combination scalpel-dilator of claim 1, wherein the grooves comprise a leading deep portion and a more shallow trailing portion running away from the tapered end.

3. The combination scalpel-dilator of claim 1, further comprising a point formed by the one or more ridges between the two or more grooves.

4. The combination scalpel-dilator of claim 1, further comprising one or more serrations in at least one of the one or more ridges.

5. The combination scalpel-dilator of claim 1, wherein the combination scalpel-dilator has two grooves and one ridge.

6. The combination scalpel-dilator of claim 1, whether each of the two or more grooves are the same length or different lengths.

7. The combination scalpel-dilator of claim 1, whether each of the two or more grooves are the same depth or different depths.

8. The combination scalpel-dilator of claim 1, wherein the combination scalpel-dilator of claim 1 comprises metal, plastic, or a combination thereof.

9. The combination scalpel-dilator of claim 1, where the outside diameter (OD) of a final dilating section of the combination scalpel-dilator is marginally smaller than the OD of a desired final device to be inserted into the tissue of a subject.

10. The combination scalpel-dilator of claim 1, further comprising a Luer-type connector on the tubular portion opposite the tapered end.

11. A kit for percutaneous placement of a cannula or other device, comprising the combination scalpel-dilator of claim 1 in sterile packaging.

12. The kit of claim 11, further comprising one or more of a guide wire, a needle, a syringe and/or a cannula or other device to be implanted in a subject, wherein the cannula or other device are sized for use with the combination scalpel-dilator.

13. A method of percutaneous placement of a cannula or other device into a blood vessel of a subject, comprising:
percutaneously entering a target blood vessel with a needle and syringe;
removing the syringe and placing a soft-tipped guide wire through the needle lumen into the lumen of the vessel and advanced past the tip of the needle;
withdrawing the needle over the wire while the wire is held in place relative to the blood vessel;
advancing the combination scalpel-dilator of claim 1 forcibly over the wire to open the tract for the cannula or other device, thereby incising the skin and subcutaneous fascia to create an opening for the cannula or other device;
removing the combination scalpel-dilator while the wire is held in place relative to the blood vessel;
advancing the cannula or other device over the wire until it is at the desired depth; and
removing the wire.

14. The method of claim 13, further comprising aspirating air from the cannula or other device and then flushing with a neutral solution.

15. The method of claim 13, further comprising securing the cannula or other device to the subject to prevent movement.

16. The method of claim 13, further comprising capping the cannula or other device.

17. The method of claim 13, further comprising aspirating blood into the syringe to confirm the needle tip is in the vessel lumen.
